(19) United States
(12) Patent Application Publication
(10) Pub. No.: US 2013/0190807 A1
(43) Pub. Date: Jul. 25, 2013

(54) METHOD FOR REUSABLE NASAL PASSAGE DILATOR

(71) Applicant: Allen Richard Andis, Racine, WI (US)

(72) Inventor: Allen Richard Andis, Racine, WI (US)

(21) Appl. No.: 13/355,702

(22) Filed: Jan. 23, 2012

(51) Int. Cl. A61F 5/08; A61M 29/00

(52) U.S. Cl. 606/204.45

(57) ABSTRACT

A method for a resuable nasal passage dilator comprising the steps of providing a flat flexible strip member with an inner surface and an outer surface and a first adhesive bandage, having an adhesive surface and a non-adhesive surface. The outer surface of the flexible strip member is secured onto the adhesive surface of the first adhesive bandage, thereby temporarily securing the flexible strip member to the bandage. The flexible strip member and the first adhesive bandage are positioned onto a user's nose. The flexible strip member and first adhesive bandage are then removed from the user's nose after a period of use and the outer surface of the flexible strip member and the adhesive surface of the first adhesive bandage are separated. The steps of the method are then repeated with a subsequent adhesive bandage and the same flexible strip member.

### FIELD OF THE INVENTION

This invention relates to nasal passage dilators and, more particularly, to methods of reusing nasal passage dilators.

### BACKGROUND OF THE INVENTION

So-called “nasal dilators,” consist of mainly two kinds of dilators, one kind which opens the nostrils from the outside and another that dilates the nostril from the inside. The external dilator typically consists of an adhesive stiff strip that is applied to the outside surface of the nose. It works by pulling the nostrils open, easing breathing through the users nose and thereby reducing snoring (or other common breathing problems).

In the nasal dilator field, there is a need for an improved external nasal dilator. External nasal dilators of the prior art have shortcomings. One such shortcoming is that such external nasal dilators if effective, are not resuable. As a result, users often wear an external nasal dilator for a few hours or overnight and then dispose of the nasal dilator in the trash. Such short use and subsequent disposal leads to an increase in the amount of trash, something which is not desirable or environmentally friendly. Another shortcoming is that typically the longer an external nasal dilator is worn the less effective the adhesive (used to secure the dilator to the user) becomes. As a result, external nasal dilators may become inadvertently removed from a user's nose.

Examples of external nasal dilators include dilators which have an stiff strip which is adhesive and is applied to the user's nose. Such external nasal dilators are usually deemed unsatisfactory because of their short usable life span and disposable nature. General examples of nasal dilators are disclosed in the following United States patents: U.S. Pat. No. 5,957,126 (Neeser), U.S. Pat. No. 5,533,503 (Doubek, et al.) and U.S. Pat. No. D380,264 (Petruson) as well as U.S. Patent Application No. 2009/0125052 (Pinna, et al.).

This invention overcomes certain problems and shortcomings in the prior art, including those mentioned above and others by namely providing a method for a reusable external nasal dilator.

### SUMMARY OF THE INVENTION

An inventive method for a reusable nasal passage dilator is disclosed. The method includes the steps of providing (a) a flat flexible strip member with an inner surface and an outer surface and (b) a first adhesive bandage, having an adhesive surface and a non-adhesive surface; securing the outer surface of the flexible strip member onto the adhesive surface of the first adhesive bandage, thereby temporarily securing the flexible strip member to the bandage, the flexible strip member being lengthwise centrally positioned on the adhesive surface; positioning and conforming. the combination of the flexible strip member and the first adhesive bandage onto a surface of a user's nose in a manner perpendicular to the ridge of the nose so that the combination of the flexible strip member and first adhesive bandage conform to the shape of the user's nose; removing the combination of the flexible strip member and first adhesive bandage from the user's nose after a period of use as determined by the user; separating the outer surface of the flexible strip member from the adhesive surface of the first adhesive bandage; and positioning the outer surface of the flexible strip member onto an adhesive surface of a second adhesive bandage for use on the surface of the user's nose for later use and removal.

It is highly preferred that the flexible strip member be reusable with different and subsequent adhesive bandages throughout the life of the flexible strip member. Preferably, the combination of the flexible strip member and the first (as well as subsequent) adhesive bandage are positioned onto the user's nose halfway between the bridge of the nose and the tip of the nose.

In preferred embodiments, the adhesive surface of the bandage includes a non-adhesive pad which aids in the removal of the flexible strip member from the adhesive surface. The inner surface of the flat flexible strip member contacts the user's nose for extended periods of time. Such usage will. negate removal of residual adhesive adhering to the users nose following use.

The term “flexible strip member,” as Used herein, refers to a flat piece of plastic, metal or composite material that is non-toxic in nature when in contact with human skin or tissue. The term “adhesive bandage,” refers to a piece of cloth or other material commonly used to bind up a wound or sore which has an area that is able to adhere to skin.

### BRIEF DESCRIPTION OF THE DRAWINGS

FIG. 1 is a perspective view of the reusable nasal passage dilator secured to a user's nose.

FIG. 2 is a further view of the reusable nasal passage dilator of FIG. 1 taken in the direction of line 2-2.

FIG. 3 is an exploded view of the reusable nasal passage dilator of FIG. 1.

FIG. 4 is a front view of the reusable nasal passage dilator of FIG. 1.

FIG. 5 is back view of the reusable nasal passage dilator of FIG. 1.

FIG. 6 is side view of the reusable nasal passage dilator of FIG. 1.

### DETAILED DESCRIPTION OF PREFERRED EMBODIMENTS

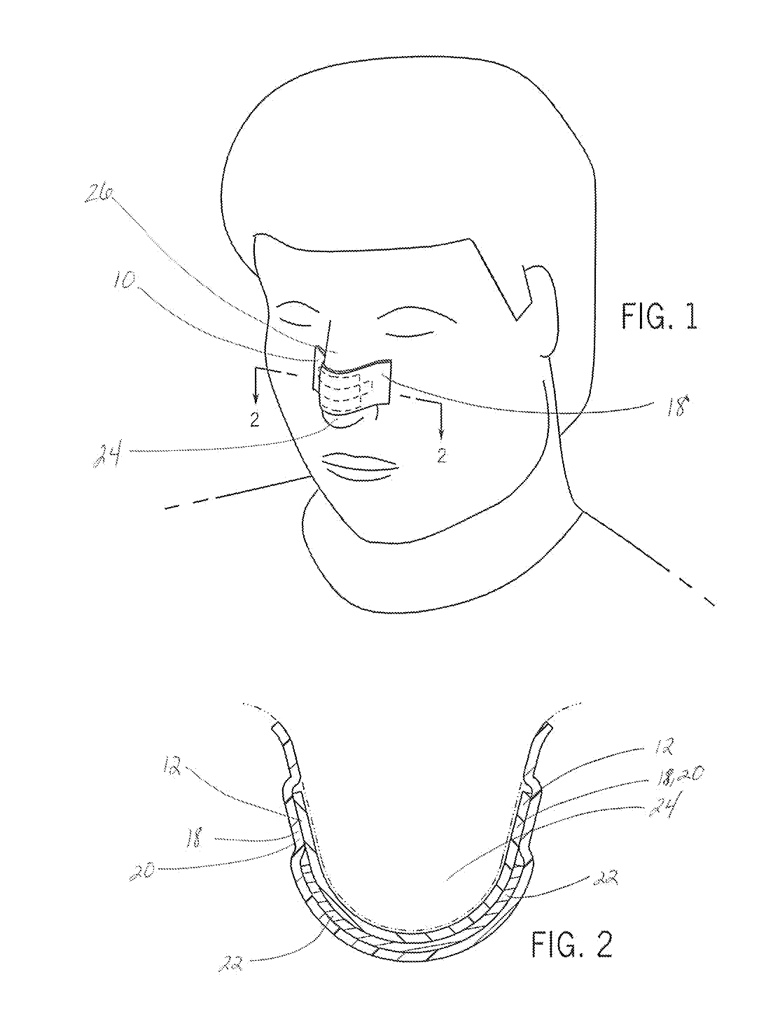

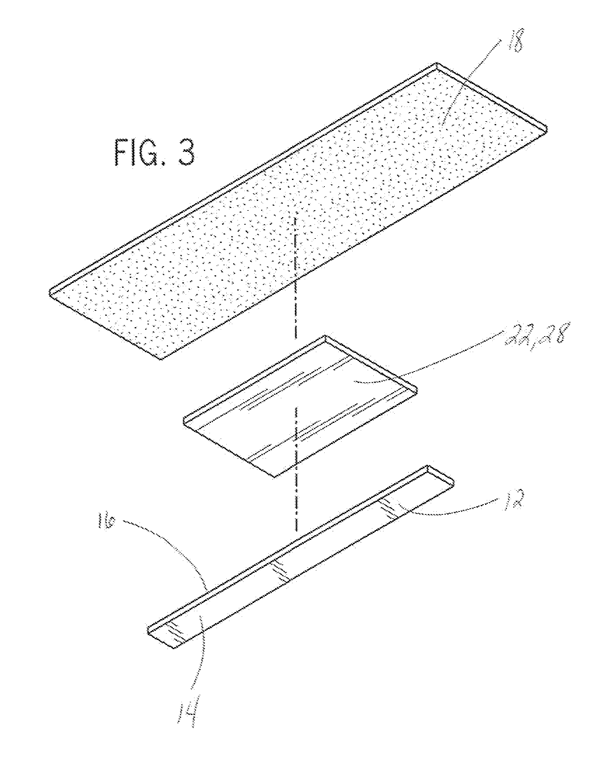

FIGS. 1-6 illustrate an inventive method for a reusable nasal passage dilator 10. The inventive method includes providing a flexible strip member 12 with an inner surface 14 and an outer surface 16, as well as providing a first adhesive bandage 18 (also generally referred to herein as “bandages”) having an adhesive surface 20 and a non-adhesive surface 22. The inventive method includes the outer surface 16 of flexible strip member 12 being secured onto adhesive surface 20 of first adhesive bandage 18, thereby temporarily securing flexible strip member 12 to the bandage 18, flexible strip member 12 being lengthwise centrally positioned on the adhesive surface 20.

Next, flexible strip member 12 and the first adhesive bandage 18 are positioned and conformed to a surface of a user's nose 24 in a manner perpendicular to the ridge of the nose 26 so that the combination of flexible strip member 12 and first adhesive bandage 18 conform to the shape of user's nose 24 as seen in FIG. 1. Flexible strip member 12 and first adhesive bandage 18 are then removed from user's nose 24 after a period of use as determined by user and outer surface of flexible strip member 12 is separated from adhesive surface 20 of first adhesive bandage 18. Outer surface 16 of flexible strip member 12 is then positioned onto adhesive surface 20 of a second adhesive bandage 18 for use on the surface of user's nose 24 for later removal after use has been completed.

The above method can be repeated using as many adhesive bandages 18 as needed over the life of flexible strip member 12. Reusable nasal dilator 10 allows reuse of a single flexible strip member 12, which is preferably a flat spring-type strip, by attaching it to subsequent adhesive bandages 18. Flexible strip member 12 is preferably a flat piece of plastic, metal or composite material that is non-toxic in nature when in contact with human skin or tissue. Flexible strip member 12 is preferably of a thickness to be flexible enough to comfortably conform to the shape of a user's nose 24. First adhesive bandage 18 and subsequent adhesive bandages 18 can be the same type of bandage or each can vary in size and dimension depending on the needs of the user.

Flexible strip member 12 is reusable with different adhesive bandages 18 throughout the life of flexible strip member 12. Bandages 18 of this type are widely available over the counter at numerous retail stores and are inexpensive. Bandages 18 of this type have been used by most segments of the population and their application in the treatment of wounds and sores is quite common.

In some alternative embodiments, flexible strip member 12 can be reduced in length, width or both dimensions to customize reusable nasal dilator 10 for individual needs and to accommodate the numerous sizes of adhesive bandages 18 available on the market.

Flexible strip member 12 and first adhesive bandage 18 are positioned onto user's nose 24 halfway between the bridge of the nose, and the tip of the nose. FIG. 1 illustrates, reusable nasal passage dilator 10 on the nose 24 of a user. The adhesive bandage 18 in FIG. 1 is representative of a first adhesive bandage 18, second adhesive bandage 18 and subsequent adhesive bandages 18.

As seen in FIG. 2, outer surface 16 of flexible strip member 12 is secured onto adhesive surface 20 of an adhesive bandage 18. FIG. 2 also illustrates that it is preferable that flexible strip member 12 be lengthwise centrally positioned on adhesive surface 20 of adhesive bandage 18.

As seen best in FIGS. 2-4, adhesive surface 20 has a non-adhesive surface which includes a non-adhesive pad 28 which aids in the removal of flexible strip member 12 from adhesive surface 20. Most adhesive bandages 18 include a non-adhesive pad 23 (also commonly referred to as a “wound pad”) which prevents flexible strip member 12 from becoming imbedded in adhesive for its entire length. This is advantageous since it minimizes stress damage to flexible strip member 12 during its removal because strip member 12 is only adhered to adhesive bandage 18 at the very ends of strip member 12. Non-adhesive pad 28 also makes removal of bandage 18 from flexible strip member 12 easier. No tools are needed to aid in the removal of bandage 18 from flexible strip member 12. Minimal hand-eye coordination is all that is needed.

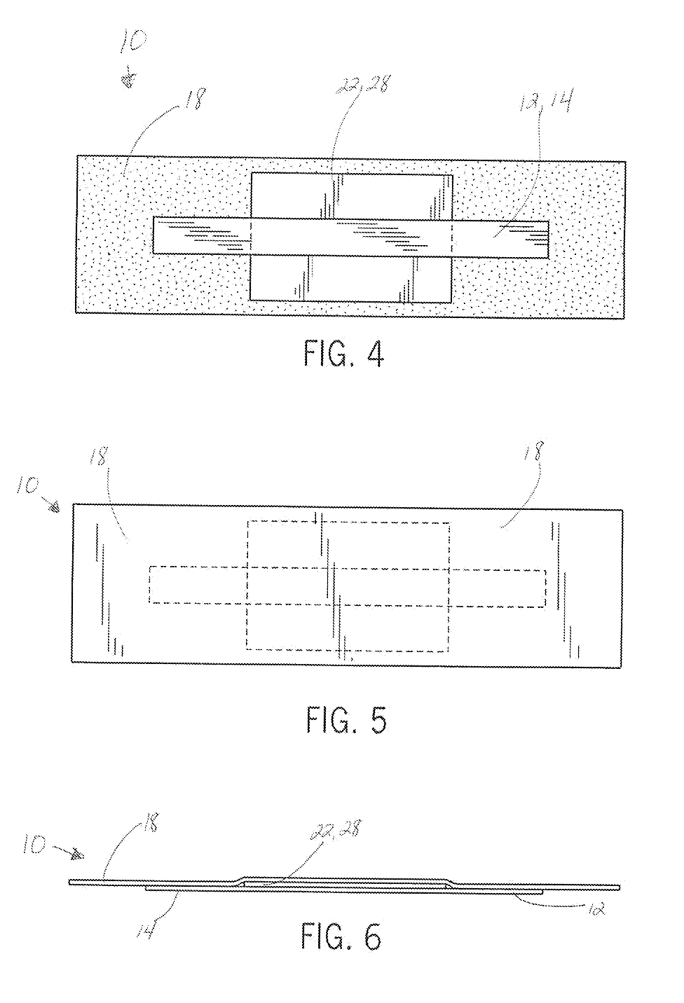

FIG. 4 illustrates inner surface 14 of flexible strip member 12. Outer surface 16 of flexible strip member 12 is the portion that contacts non-adhesive surface 22 of adhesive bandage 18 as seen in FIGS. 4-5. FIG. 5 also illustrates the portion of adhesive bandage 18 which does not directly contact user's nose 24.

FIG. 6 illustrates the interaction between adhesive bandage 18, non-adhesive surface 22, adhesive surface 20 and their interaction with flexible strip member 12. As illustrated in FIGS. 1-2 and 6, the overall width of reusable nasal dilator 10 is minimal. The minimal width of reusable nasal dilator 10 and the ability of dilator 10 to conform to a user's nose 24 make dilator 10 comfortable to use.

It is common and expected that inner surface 14 of flexible strip member 12 may contact user's nose 12 for extended periods of time, such as overnight while user is sleeping. Reusable nasal dilator 10 provides a simple, low-cost and easily available method for opening up nasal passages with low environmental impact.

While the principles of this invention have been described in connection with specific embodiments, it should be understood clearly that these descriptions are made only by way of example and are not intended to limit the scope of the invention.

1. A method for a resuable nasal passage dilator comprising the steps of:
providing (a) a flexible strip member with an inner surface and an outer surface and (b) a first adhesive bandage, having an adhesive surface and a non-adhesive surface;
securing the outer surface of the flexible strip member onto the adhesive surface of the first adhesive bandage, thereby temporarily securing the flexible strip member to the bandage, the flexible strip member being lengthwise centrally positioned on the adhesive surface;
positioning and conforming the flexible strip member and the first adhesive bandage onto a surface of a user's nose in a manner perpendicular to the ridge of the nose so that the flexible strip member and first adhesive bandage conform to the shape of the user's nose;
removing the flexible strip member and first adhesive bandage from the user's nose after a period of use as determined by the user;
separating the outer surface of the flexible strip member from the adhesive surface of the first adhesive bandage; and
positioning the outer surface of the flexible strip member onto an adhesive surface of a second adhesive bandage for use on the surface of the user's nose for later use and removal.

2. The method of claim 1 wherein the flexible strip member is reusable with different adhesive bandages throughout the life of the flexible strip member.

3. The method of claim 1 wherein the flexible strip member and the first adhesive bandage are positioned onto the user's nose halfway between the bridge of the nose and the tip of the nose.

4. The method of claim 2 wherein the flexible strip member and adhesive bandage are positioned onto the user's nose halfway between the bridge of the nose and the tip of the nose.

5. The method of claim 1 wherein the adhesive surface includes a non-adhesive pad which aids in the removal of the flexible strip member from the adhesive surface.

6. The method of claim 1 wherein the inner surface of the flat flexible strip member contacts the user's nose for extended periods of time.
